(19) United States
(12) Patent Application Publication
(10) Pub. No.: US 2007/0083144 A1
(43) Pub. Date: Apr. 12, 2007

(54) Blood irradiation system device

(76) Inventor: Thomas R. Petrie, Newfoundland, NJ (US)

Correspondence Address: MINTZ LEVIN COHN FERRIS GLOVSKY & POPEO, 666 THIRD AVENUE, NEW YORK, NY, 10017, US

(21) Appl. No.: 11/441,547

(22) Filed: May 26, 2006

Related U.S. Application Data
(63) Continuation-in-part of application No. 11/285,959, filed on Nov. 22, 2005.
(60) Provisional application No. 60/685,471, filed on May 27, 2005.
(60) Provisional application No. 60/630,503, filed on Nov. 22, 2004.
(60) Provisional application No. 60/638,286, filed on Dec. 21, 2004.

(51) Int. Cl. A61M 37/00

(52) U.S. Cl. 604/6.08

(57) ABSTRACT

Embodiments of the present application are directed to devices, systems and methods for irradiating fluids (e.g., blood) with ultraviolet light, and corresponding related components, systems and methods. In some embodiments of the invention, an ultraviolet blood irradiation (UBI) system is provided and may include an ultraviolet UV source providing a predetermined wavelength of radiation to provide a detrimental effect to virus and/or bacteria, an exposure chamber for exposing a predetermined volume of blood to radiation, a conduit between the UV source and the exposure chamber, a pump for pumping blood between a first location and a second location and a shutter assembly provided between the UV source and the exposure chamber providing time-metered irradiation of the blood in the chamber.

### CLAIM TO PRIORITY AND CROSS-REFERENCED APPLICATIONS

The present application claims priority under 35 U.S.C. § 119(e) to U.S. Provisional Patent Application No. 60/685,471 to Petrie, filed May 27, 2005, and titled “Blood Irradiation Device” and disclosure of which is incorporated herein by reference in its entirety.

The present application is also a continuation-in-part application of U.S. patent application Ser. No. 11/285,959 to Petrie, filed Nov. 22, 2005, which claims priority under 35 U.S.C. §119(e) to U.S. provisional application nos. 60/630,503, filed Nov. 22, 2004 and 60/638,286, filed Dec. 21, 2004. The present application is related to U.S. Pat. No. 6,312,593 to Petrie. Each of the foregoing disclosures is herein incorporated by reference in their entirety.

### BACKGROUND OF THE INVENTION

1. Field of the Invention

Embodiments of the present invention are directed to devices, systems and methods for irradiating fluids (e.g., blood) with ultraviolet light, and corresponding related components, systems and methods.

2. Background of the Invention

It has long been recognized and understood that specific wavelengths of ultraviolet radiation have the ability to destroy certain biological and chemical structures. While the sun and most active celestial bodies normally emit all types of UV radiation, portions of the earth's atmosphere prevent its destructive form of energy from reaching the surface.

During the last century, scientists and medical practitioners experimented with the use of UV radiation in the treatment of diseases. One such experiment in the late 1930's involved the development of a rudimentary device designed to expose human blood to a UV lamp, in an effort to kill virus and bacteria. This particular device, while medically successful with respect to the patients being treated, was an electrical and mechanical failure due to several factors. First and foremost, the UV lamp was difficult to operate; just to get the lamp to strike was a major handling problem. There were numerous interactive controls that required constant re-adjustment to keep the device operating properly. In addition, the lamp had only a short lifespan before it either failed to strike, or produce the necessary therapeutic wavelength of UV. There was also an ongoing general maintenance issue with a water cooling process and a belt drive sequence of included mechanics. In addition, the control of the flow rate of the blood through the system also required constant adjustment and monitoring by a trained operator. Because of the design of the device, blood collection was also difficult. Specifically, gravity was used to draw and collect the blood into an open beaker. The beaker was than moved to a position above the device and allowed to drain through the pump and exposure chamber.

Although positive therapeutic treatments resulted when all system components were operating properly, such conditions did not occur often. Moreover, if a mechanical, electrical or lamp problem developed during the course of a clinical procedure, the system provided no visual or audible indications to notify the operator or an automatic fail-safe termination of operation.

### SUMMARY OF THE INVENTION

Accordingly, in response to the problems of such prior art systems and devices for blood irradiation, embodiments of the present invention are provided. While preferred embodiments of the present invention utilize the same fundamental principal to irradiate blood, such embodiments provide a dramatically improved system and process. In some embodiments, the system automatically controls and monitors the blood irradiation process. Moreover, embodiments of the present invention may include established clinical parameters to ensure a safe and therapeutically effective medical procedure.

In an embodiment, the present invention is an exposure chamber for exposing blood to radiation in a blood irradiation system having an ultraviolet UV source, a connector between the UV source and the exposure chamber, a pump for pumping blood through the exposure chamber, and a shutter assembly provided between the UV source and the exposure chamber for time-metered radiation of the blood in the exposure chamber. The exposure chamber includes a housing including an entry conduit and an exit conduit; a UV filter lens configured to be secured within the housing; a gasket configured to be secured within the housing and substantially adjacent to the UV filter lens. The gasket includes openings configured to be in communication with the entry conduit and the exit conduit of the housing. The gasket includes an insert configured to create an exposure area. The exposure area is further configured to be in communication with the openings of the gasket. The entry and exit conduits, the openings, and the exposure area are configured to create a channel for permitting blood flow through the exposure chamber.

In an alternate embodiment, the present invention is an exposure chamber for exposing blood to radiation in a blood irradiation system having an ultraviolet UV source, a connector between the UV source and the exposure chamber, a pump for pumping blood through the exposure chamber, and a shutter assembly provided between the UV source and the exposure chamber for time-metered radiation of the blood in the exposure chamber. The exposure chamber includes a housing including a conduit; another housing including another conduit, wherein the housing is configured to be coupled to the another housing; a gasket configured to be secured between the housing and the another housing and including openings configured to be aligned with the conduit and the another conduit; a filter lens configured to be secured between the housing and the gasket; another filter lens configured to be secured between the another housing and the gasket; an insert and another insert configured to be secured within the gasket, the inserts form an exposure area within the gasket. The conduits, the openings and the exposure area form a channel configured to allow blood to flow through the exposure chamber.

In another alternate embodiment, the present invention is a micro-channel assembly for allowing blood to flow through an exposure chamber for exposing blood to radiation in a blood irradiation system having an ultraviolet UV source, a connector between the UV source and the exposure chamber, a pump for pumping blood through the exposure chamber, and a shutter assembly provided between the UV source and the exposure chamber for time-metered radiation of the blood in the exposure chamber. The micro-channel includes a conduit configured to be secured within a housing of the exposure chamber; an opening within a gasket of the exposure chamber and configured to be in communication with the conduit; an exposure area within the gasket and configured to communicate with the opening; another opening within the gasket and configured to communicate with the exposure area; another conduit secured within the housing and further configured to communicate with the another opening.

These and other embodiments, features, advantages and objects of the invention will become even more apparent with reference to the following detailed description and attached drawings, a brief description of which is set out below.

### BRIEF DESCRIPTION OF THE DRAWINGS

FIG. 1 is a perspective view of a blood irradiation system according to some of the embodiments of the present invention.

FIG. 2 is a side view of an exemplary UV source (e.g., lamp/bulb) according to some of the embodiments of the present invention.

FIG. 3 is a spectral output of a UV source used in some embodiments of the present invention.

FIG. 4 is a schematic side view of an administration set according to some embodiments of the present invention.

FIG. 5 is a perspective view of an exposure chamber according to some of the embodiments of the present invention.

FIG. 6 is a front view of a reservoir/flow-through bag according to some of the embodiments of the present invention.

FIG. 7 is an enlarged portion “A”, as depicted in FIG. 6, of the reservoir/flow-through bag.

FIG. 8 is an enlarged portion “AA”, as depicted in FIG. 7, of the reservoir/flow-through bag.

FIG. 9 is an exploded perspective view of a blood irradiation system according to some embodiments of the present invention.

FIG. 10 is an exploded perspective view of an interior portion of a blood irradiation system according to some embodiments of the present invention.

FIG. 11A is an exploded perspective view of a shutter assembly according to some embodiments of the present invention.

FIG. 11B is a front view of the shutter assembly, having an exposure chamber included therein, in a “closed” position.

FIG. 11C is a front view of the shutter assembly, having an exposure chamber included therein, in an “open” position.

FIG. 12 is an exploded perspective view of a UV lamp housing according to some embodiments of the present invention.

FIG. 13 is a side, cross-sectional view of a blood irradiation system according to some embodiments of the present invention.

FIG. 14 is a front view of a chopper-wheel assembly according to some embodiments of the present invention.

FIG. 15 is a side view of a chopper-wheel assembly according to some embodiments of the present invention.

FIG. 16 is a block diagram of a blood irradiation system according to some embodiments of the present invention.

FIG. 17 is a flowchart of an operation of a blood irradiation system according to some embodiments of the present invention.

FIG. 18 is an exploded perspective view of a blood exposure chamber, according to the present invention.

FIG. 19 is an exploded perspective view of two housing portions of the blood exposure chamber shown in FIG. 18, according to the present invention.

FIG. 20 illustrates a top view of an outside surface and two cross-sectional side views of the housing portion of the blood exposure chamber shown in FIG. 18, according to the present invention.

FIG. 21 illustrates a top view of an inside surface and two side views of the housing portion of the blood exposure chamber shown in FIG. 18, according to the present invention.

FIG. 22 is a perspective view of a gasket of the blood exposure chamber shown in FIG. 18, according to the present invention.

FIG. 23 illustrates a top view and a side view of the gasket of the blood exposure chamber shown in FIG. 22, according to the present invention.

FIG. 24 illustrates a top view and a side view of an UV filter lens of the blood exposure chamber shown in FIG. 18, according to the present invention.

FIG. 25 illustrates a top view and a side view of an insert of the blood exposure chamber shown in FIG. 18, according to the present invention.

FIG. 26 illustrates an assembled blood exposure chamber shown in FIG. 18, according to the present invention.

### DETAILED DESCRIPTION OF THE EMBODIMENTS

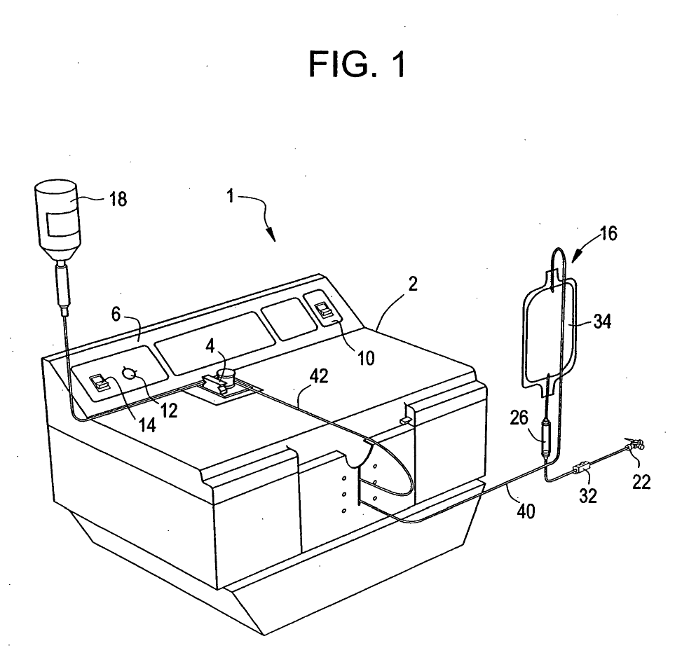

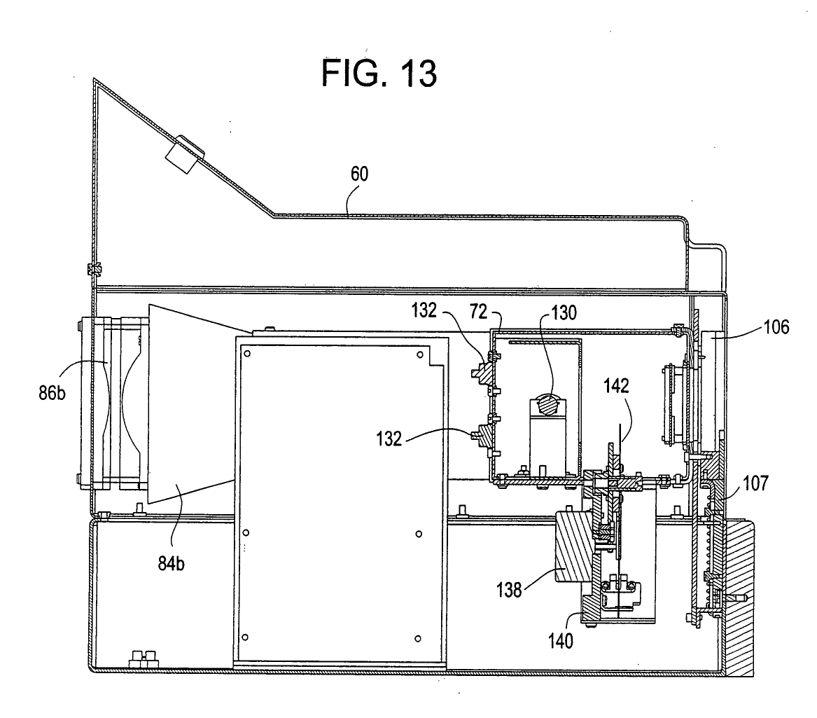

FIGS. 1-17 illustrate some of the embodiments of the present invention. To that end, some of the figures illustrate an Ultraviolet Blood Irradiation (UBI) system (also referred to as a “Hemo-Modulator”) which includes specifically arranged mechanical and electronic components to provide a well-defined exposure of patient blood to UV radiation. According to some embodiments of the present invention, such mechanical and electrical components may include an ultraviolet (UV) lamp, a fluid pump, an exposure chamber, and control logic means. Such embodiments may also include a variety of sensory items for monitoring and carrying out irradiation and other operation processes.

FIG. 1 illustrates one embodiment of the present invention directed to a UBI system for irradiating blood (or other fluid). As shown, the system is housed in a convenient cabinet 2, which may be provided as a table-top unit or may include structure with wheels (i.e., a cart), such that the cabinet/system may be easily transported. Mounted on the cabinet may be a pump 4, control panel 6 and a receiving portion/housing 8 for receiving a blood exposure chamber of an administration set (described below).

The control panel may include controls, including, for example, a switch for main power 10, UV lamp switch 12 (preferably a keyed switch), and pump switch 14. Each switch may also include one or more corresponding LED lights 18 for indicating a status of the associated mechanism (e.g., main power “on/off”). For example, the main power switch may include a red LED which is lit when the switch is in the on position. Similarly, the UV lamp switch preferably includes a series of associated LEDs for indicating a “warm-up” condition (UV lamp warming up to operating condition). For example, in a short time (e.g., between about 30-120 seconds, preferably around 90 seconds) a red LED may be lit upon initial lamp turn-on (indicating that the lamp is not yet ready to irradiate blood) which may then turn off upon the lamp reaching an operating condition—at that point, a green LED may be lit (or the red LED may change to green) indicating that the UV lamp is ready to irradiate blood. To ensure a long lifespan of the UV lamp, the lamp preferably is turned on and off as little as possible. Thus, if a plurality of patients require treatment, the UV lamp preferably remains “on” the entire time (e.g., left “on” between individual blood irradiations).

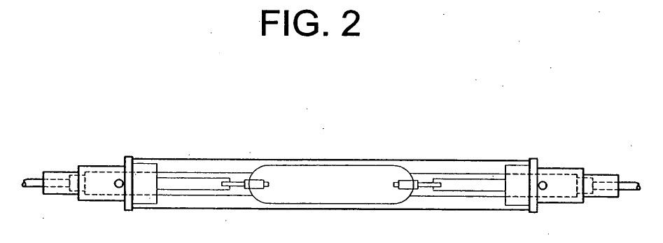

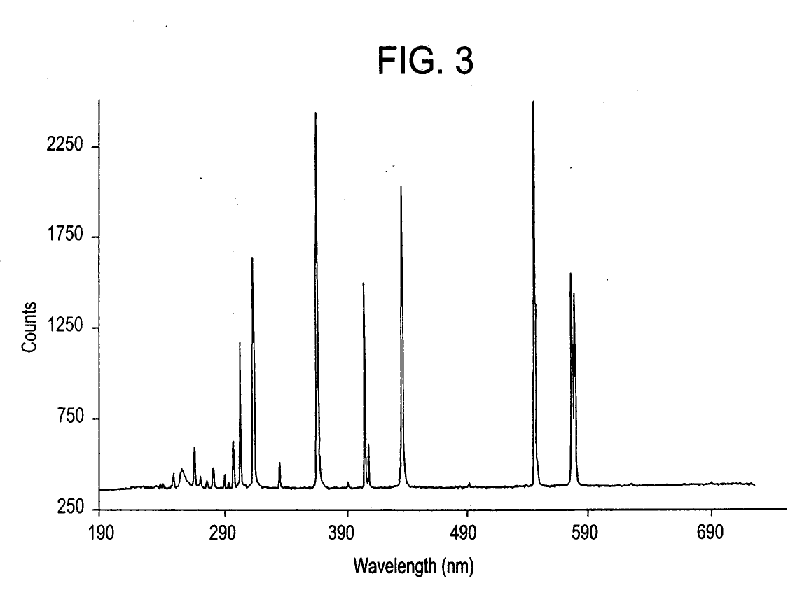

The UV lamp preferably provides a specific wavelength of radiation known to be clinically effective in destroying or substantially destroying virus and/or bacteria. Such wavelengths may be between 200-400 nm, to treat, for example, human immunodeficiency virus (HIV-1, HIV-2), autoimmunodificiency syndrome (AIDS) in human and animal whole blood, blood products and process blood components. The UV lamp may be encased in a glass tube to stabilize and maintain proper operating temperature and eliminate any foreign matter contact. While in one embodiment of the invention, the UV lamp may comprise a 210 watt medium pressure mercury vapor lamp, having 2.0″ arc, an overall length of about 8.5 inches and a width of about 1.0 inch, other types of UV lamps of different wattages, lengths, widths and arcs may be used. One of skill in the art will appreciate that a change in the bulb, to a different type. FIG. 2 represents a side view of one type/size of UV lamp that may be used in embodiments of the present invention. FIG. 3 represents the relative spectral and energy output of such a lamp.

Pump 4 is used to flow blood, at predetermined flow rates, through the exposure chamber, and preferably in both directions. In preferred embodiments, the pump comprises a peristaltic pump, although other types of pumps may be used. The flow rate of the pump may depend on an assortment of variables including UV lamp strength, exposure chamber design and/or volume, and the size/diameter of the tubing/conduit (i.e., PVC or silicon tubing) which transports the blood to and from the pump and/or exposure chamber. The pump fluid flow rate is preferably is set to a predetermined calibrated flow, but some embodiments of the invention may include controls as to adjust the flow rate to a number of settings. Typically, the predetermined set flow rate may be routinely checked to ensure proper operation of the system. Such inspection may be accomplished via a visual flow indicator (e.g., flow gauge). Commercially available flow rate sensors may be included to monitor the flow rate and initiate a shut down of the system upon the rate varying greater than a predetermined amount (e.g., plus or minus 5 percent of the ideal flow rate). Such monitoring may be effected by an electrical/computer control system (for example).

FIG. 1 also illustrates the UBI system with administration set 16 connected thereto. According to some embodiments of the invention, the administration set is a single-use, disposable system. This ensures that blood from one patient does not mix with blood from another patient, and allows the system to operate inexpensively and effectively. As shown, blood from a patient is collected in a collection reservoir 18 (IV bottle/bag). A portion of tubing of the administration set is placed in the peristaltic pump so that the pump can act on the tubing to create a pumping pressure in one or preferably both directions, depending upon whether blood is being sent to or from the reservoir container.

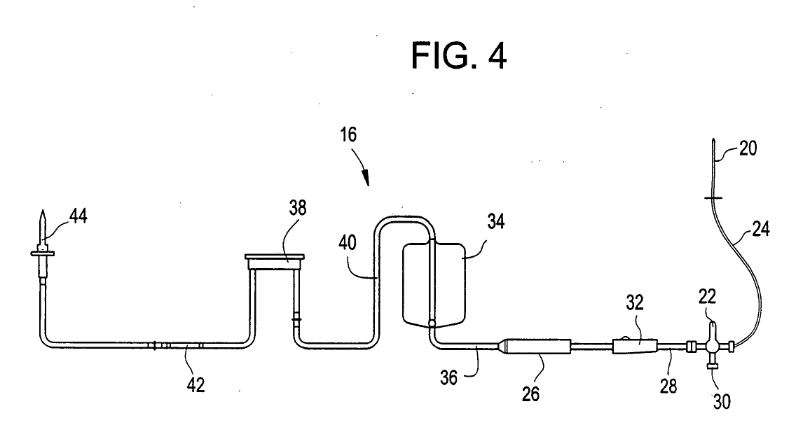

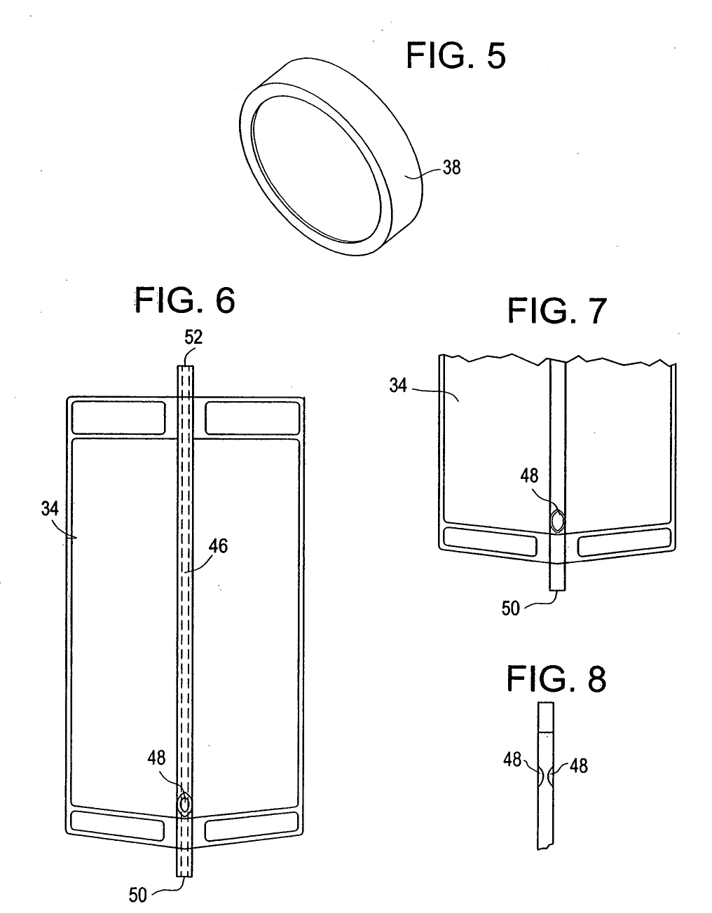

As shown in FIG. 4, some preferred embodiments of the systems administration set include a needle 20 for insertion into a patient for collecting and infusing blood, this may be directly connected to a three-way stopcock 22, or a length of PVC/silicon tubing 24 may connect the stopcock and needle. The stopcock is connected to a drip-tube via PVC tubing 28. The three-way stopcock may include a port for transfer of fluids to a patient (patient port) being connected to a patient needle, another port 30 for a syringe (syringe port) and the third port for communicating fluids to/from the system. Along the PVC tubing connecting the stopcock to the drip-tube may be a roller clamp 32 (or other clamp) to stop flow of blood to/from the patient. The drip-tube may then be connected to a soft walled venous reservoir 34 via a length of PVC tubing 36, which may then be connected to one side of exposure chamber 38 (see also, FIG. 5) via PVC tubing 40.

A length of silicone tubing 42 (which may be used in combination with the peristaltic pump) is connected from the other side of the exposure chamber to a blood spike 44 for insertion into an IV bottle (vacuum bottle; e.g., Vac Bottle 500 ml by McGaw). While silicone tubing may be used along the entire length from the UV exposure chamber to the blood spike, PVC tubing may be used as well or a combination thereof.

The soft wall venous reservoir bag 34 is shown in FIG. 6-8. In some embodiments of the present invention, the soft wall bag preferably includes a plastic tube 46, within the bag, having sufficient stiffness as to not collapse upon a vacuum being applied to the tube. The tube 46 preferably includes an opening 48 (or a plurality of such openings) at a base position of the bag 34, near a first opening 50 of the tube for ferrying liquids through the tube. A second opening 52 located at an opposite end of the tube may also be provided for ferrying liquids through the tube. Upon a vacuum (i.e., a lower pressure) being applied to the bag, a portion of a wall(s) of the bag collapses onto opening 48, which allows fluid to flow through tube 46 in the direction of the lower pressure—i.e., if a vacuum is being applied at opening 52, fluid flows from opening 50 to opening 52; if a vacuum is being applied at opening 50, then the flow is the reverse. The bag may also act as a reservoir (i.e., fill with a fluid) for a fluid being ferried (e.g., blood) upon a normal (e.g., around atmospheric) or positive pressure being applied to the bag. Thus, opening 48, in combination with the bag, operates as a valve depending upon whether positive or negative pressure is supplied to the bag.

Other embodiments of the venous reservoir bag may include a hard-walled bag which includes a tube having a valve provided at a base portion of the tube. Contrary to the embodiments described immediately above, in these embodiments, the opening at the base of the tube does not require a wall(s) of the bag to cover the hole when a negative pressure is applied to the tube. Instead, a mechanical valve located proximate the opening in the tube opens and closes the opening based on a positive (open position) or negative (closed position) pressure. Such a mechanical valve may simply comprise a “flap” of plastic (e.g., thin sheet of plastic) affixed to or near a side of the opening which, upon a negative pressure, the flap covers and substantially seals the opening, and upon a positive pressure, fluids/blood can pass through the opening to be stored (e.g., temporarily) in the bag. Other types of valves may also be used, including, for example, a ball-in-cage valve.

Accordingly, the bag performs as a conduit when a vacuum is applied when a patient's blood is being drawn and as a reservoir to collect the blood volume difference between a treatment flow rate and the patient site return rate, when blood (treated or untreated) is re-infused into the patient. Accordingly, in some embodiments of the invention, the design of bag xx minimizes hemolysis in either flow direction and allows collection of returning blood in a bulk format. For operator convenience, the bag may include a volume indication on one or both sidewalls and the bag may be used in conjunction with the treatment of whole blood or blood products.

The drip tube 26 of the administration set may be used to regulate the flow rate being applied by the vacuum and needle bore during blood collection or regulate flow for blood re-infused into a patient. The vacuum pressure may be established via a vacuum being present in the vacuum bottle (which is then transferred to the bag 34 upon blood spike 44 being inserted into the vacuum bottle), or any other way (e.g., via the pump or syringe). In some embodiments of the invention, a typical draw flow rate is approx. 25-30 ml per minute and a typical flow return rate is preferably about 10-20 ml per minute. Generally, the draw of blood from a patient may vary depending on the patient.

Typically, re-infusion flow rates are generally limited to approximately the flow rate(s) disclosed above (or similar flow rates disclosed in the prior art and/or familiar to those of skill in the art). Higher re-infusion flow rates can cause a great deal of discomfort. The re-infusion rate of blood may be regulated by an operator/doctor/nurse using a visual indication of the drip tube (for example) and an IV valve. In most cases, the irradiation process is preferably completed before all the blood is returned to the patient. To that end, the reservoir bag allows the patient to be removed from the UBI system and relax at another location while the rest of the patient's irradiated blood is returned to the patient. This frees the UBI system to perform additional treatments on other patients. In some embodiments, if managed properly, the one patient/treatment may be effected about every 12 minutes. This time may be shorter or longer depending upon flow and draw rates, and rates of irradiation (e.g., upon different diameter tubes being used, upon different dosages of radiation, and the like).

Prior to blood being returned to the patient after irradiation, the vacuum may be vented to the atmosphere. This may be done via the stopcock or any other way familiar to those of skill in the art. Accordingly, the loss of vacuum allows the soft walls of the reservoir bag 34 to relax, which allows the returning blood to accumulate/pool in the bag 34 (i.e., performing as a reservoir).

The exposure chamber 38, one embodiment of which is illustrated in FIG. 5, may also be considered part of the administration set (and thus is also preferably a single-use, disposable unit), or may be considered a separate component thereof. The exposure chamber may include two openings, which allows blood to flow from one side of the device to the other, and also preferably allows the chamber to be substantially filled with blood. The chamber preferably includes one or more protuberances to cause fluidic turbulence to the blood flow. The turbulence to the blood flow allows a more complete exposure to the UV radiation source. The exposure chamber may also include a quartz cover on one side (e.g., a side being exposed to the UV radiation), which is preferably transparent to UV in the range of about 1400 to about 4000 Angstroms or between about 140 to about 400 nm. The chamber is preferably disposable, and thus, is preferably designed for easy installation and removal from the system. An example of such a chamber may be found in issued U.S. Pat. No. 6,312,593, to Petrie, the entire disclosure of which is herein incorporated by reference.

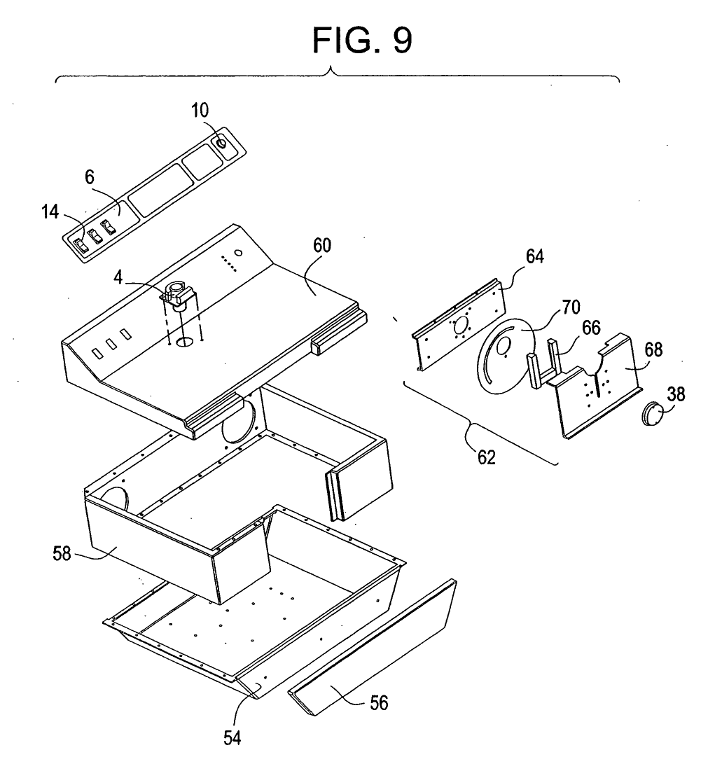

FIG. 9 illustrates an exploded perspective view of the UBI system according to some embodiments of the present invention. As shown, the cabinet xx may include a cabinet base 54, a front bumper 56, a middle wrap 58 and a cover 60. Pump 4 and control panel 6 may also be seen in this figure, as well as an exposure chamber housing assembly to house an exposure chamber 38. The exposure chamber housing may include a chamber mount 64, a shutter assembly 62, a chamber bracket 66, and a front panel 68.

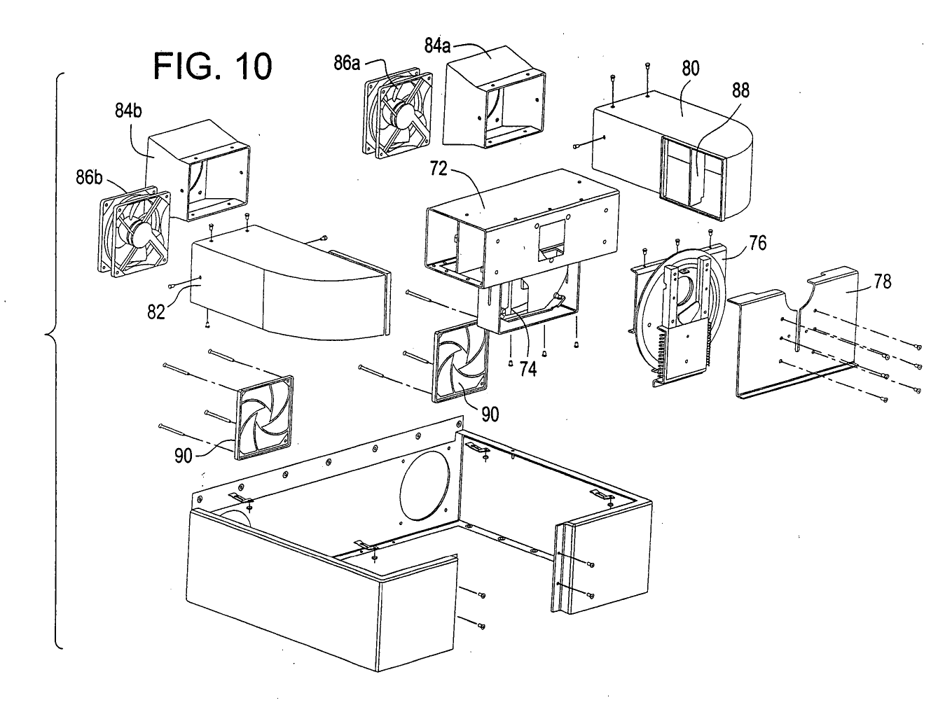

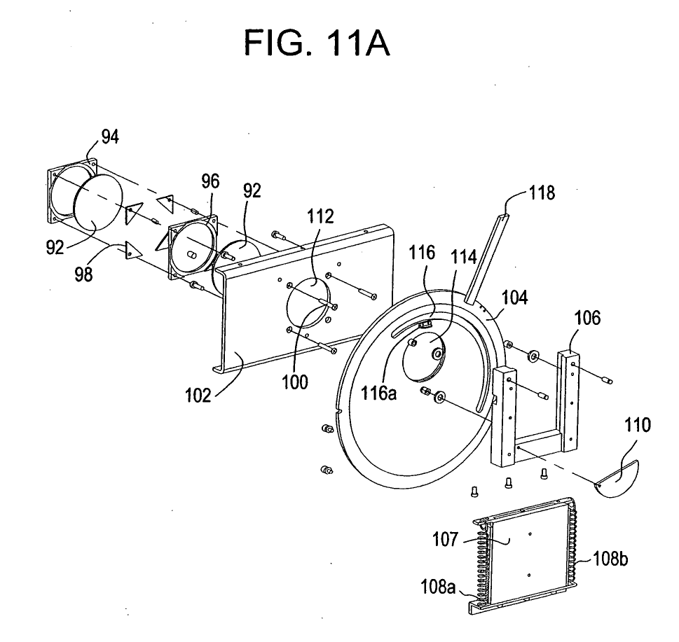

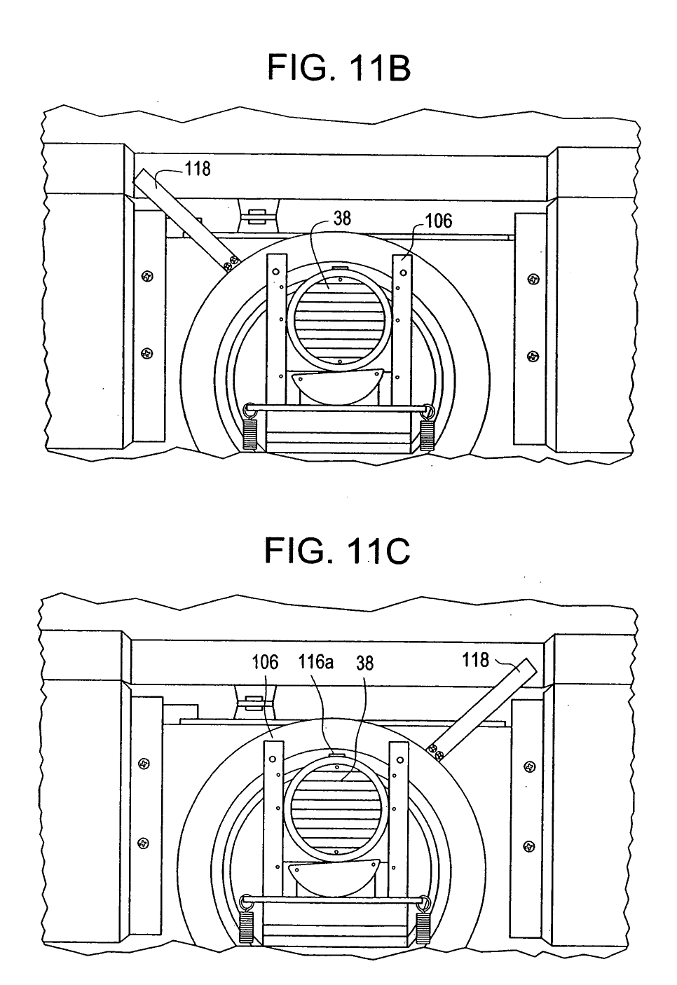

FIG. 10 is an exploded perspective view of the components housed in the center wrap section of the UBI system according to some embodiments of the present invention. Reference is also made to FIGS. 11-13. As shown, housed in the middle wrap section maybe a UV lamp housing 72 (which houses the UV lamp), chopper-wheel assembly 74 and shutter assembly 76, which also includes front panel 78. Preferably, the UV lamp housing is provided with an air cooling plenum, or is part of an air cooling plenum, which may be provided in the center wrap section. The plenum may be used to or aid in maintaining a proper temperature in the space local to the UV lamp so that the UV lamp does not overheat (causing shutdown to the system). Such a plenum may include ducts 80 and 82, each respectively connected to a duct end/shroud 84a, b.

Between each shroud (or at least one of the shrouds) and a respective duct may be a fan unit 86a, b (although the fan may be located in other areas of the middle wrap section or other portion of the cabinet/tUBI system). Each duct may include a deflector 88, to deflect all or a portion of the airflow in a predetermined direction, and/or to split the airflow. As shown in the figure, the deflector may be positioned in the center of the opening of the end of the duct which is connected to the UV lamp housing, so that the lamp receives a portion of the airflow and the chopper-wheel mechanism receives a portion. A filter 90a, b is preferably at the end of one or both of the shrouds (depending upon airflow direction). Preferably, the filters are replaceable, and conveniently positioned on a portion of the cabinet which is easily accessible (for ease of replacement).

Airflow through the UV lamp assembly may be in one direction, flowing into shroud 84a pushed by fan 86a, and exiting out shroud 84b (pulled by fan 86b). Alternatively, the flow of air may be into the UV lamp housing from both ducts; that is, fan 86a and fan 86b both draw air into each shroud, and each duct directs the air into the UV lamp housing. In the later case, a vent may be provided which allows air to vent out of the interior of at least one of the UBI system (as a whole), the lamp housing and the middle wrap section.

FIG. 11A illustrates an exploded view of the shutter assembly according to some embodiments of the present invention, which controls whether UV radiation is provided to the exposure chamber. The shutter assembly may include filter(s) 92, which may be used to filter out specific wavelength of the electromagnetic spectrum, and may be housed by filter brackets 94 and 96 (as well as other structure, e.g., clips 98 and fasteners 100). The shutter assembly may also include a chamber mounting plate 102, shutter plate assembly 104, chamber bracket 106, chamber lock assembly 107 (having springs 108a, b) and cell release cam 110. The chamber bracket is slidably connected to the chamber lock assembly, and the top of springs 108a, b attach to the bottom of the chamber bracket and the bottom of the springs are attached to the bottom of the chamber lock assembly.

The chamber mounting plate includes an opening 112, for allowing UV radiation to pass. The shutter plate assembly may include a corresponding opening 114 to allow the radiation received via opening 112 in the chamber mounting plate to pass. The shutter plate assembly may also include an elongated radial arc 116 which is slidably connected to the upper portion of chamber bracket 106.

The shutter plate assembly may also include a cam lever 118 which allows an operator to manually open and close the shutter upon the insertion of or removal of an exposure chamber. It will be appreciated by one of ordinary skill in the art, that such manual operation may be replaced by a servo or other mechanical or electromechanical device, which opens and closes the shutter according to operational parameters and/or switches located on the control panel (or located adjacent to the shutter assembly). Insertion of the chamber into the chamber receiving window results in the exposure chamber being pushed down (by the operator, for example) to release the locking cam. The cam lever may then be moved from right to left to lock the chamber into position and, in some embodiments, at the same time the exposure window is opened.

In some embodiments, movement of the cam lever 118 causes protrusion 116a to contact the upper portion of an exposure chamber inserted into the chamber bracket, and ride along an exterior diameter of the exposure chamber while also causing the exposure chamber to be pushed downward. This in turn causes the bottom portion of the exposure chamber to actuate cell release cam 110, which in turn. Pushes downward on the top portion of the chamber lock assembly. This causes the chamber bracket to rise up relative to the chamber lock assembly (i.e., the springs are stretched), to a maximum point when protrusion 116a is in a 12 o'clock position. This occurs when lever is swung to one side (“aperture open” position). To release the exposure chamber, the lever is moved to the opposite side, such that protrusion 116a no longer engages the exposure chamber and chamber bracket 106 moves downward.

Accordingly, when lever 118 is in the “open” position (see FIG. 11C; lever 118 swung to a right-side position), the exposure chamber is aligned for proper exposure to the UV radiation and prevents the escape of radiation from the front of the UBI system. When lever 118 is in the “closed” position (see FIG. 11B; lever 118 swung to a left-side position), the exposure chamber may be removed from the UBI system. Thus, movement of lever 118 in one direction or another effects and an open or a closed position: i.e., opening 114 in the shutter plate assembly moves to a position either corresponding to the opening 112 in the chamber mounting plate, or a position in which opening 114 in the shutter plate assembly does not overlap the opening 112 in the chamber mounting plate (of course, other “partial open” positions are possible, depending upon the particular use of the UBI).

A center portion of the shutter plate assembly is preferably made of polybetrafluoroethylene or may also be made of Teflon®, as may other structures of the shutter assembly which are exposed to the UV radiation. The polybetrafluoroethylene is preferable as this material is better able to withstand the repeated exposure to UV radiation, which has a detrimental effect, over time, to many materials.

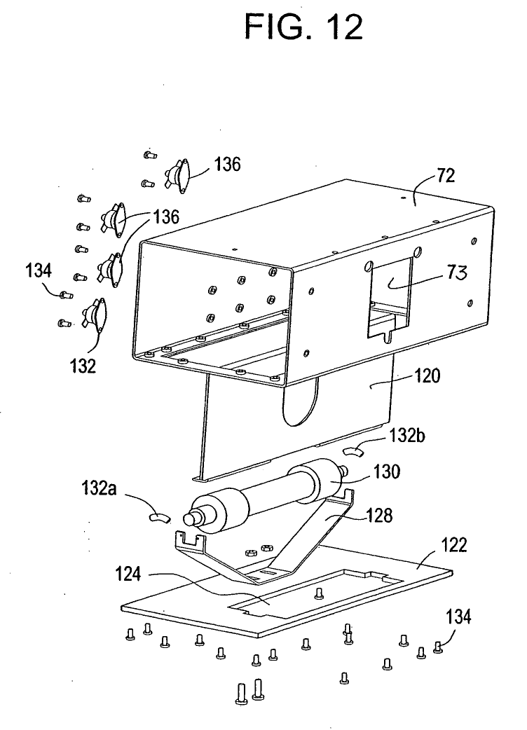

An exploded perspective view of UV lamp housing 72 is illustrated in FIG. 12. As shown, the lamp housing includes internal light port 120, chopper assembly plate 122 (with opening 124 for allowing chopper wheel 126 to pass therethrough), lamp bracket 128, lamp 130, springs 132a, b. A plurality of fasteners 134 may be used to assemble one or more of the components. Upon the air plenum (see above) failing to keep the UV lamp cool, one or more thermally sensitive electrical circuit breakers 136 (e.g., one or more thermisters) may be included to shut down the UV lamp (but preferably keep the air plenum operational). Such thermisters may be located in series on the exterior of a portion of the air plenum across from the UV lamp and may operate to turn the lamp off and prevent any restart if the plenum surface temperature reaches 50° C. (for example). In another exemplary embodiment, the temperature can reach up to 90° C. Such a series wired dual thermistor design may be used as a redundant safety system design for additional protection. These features aid in ensuring a long life, high number of turn-on strikes, and a stable and repeatable ultraviolet wavelength of radiation from the UV lamp.

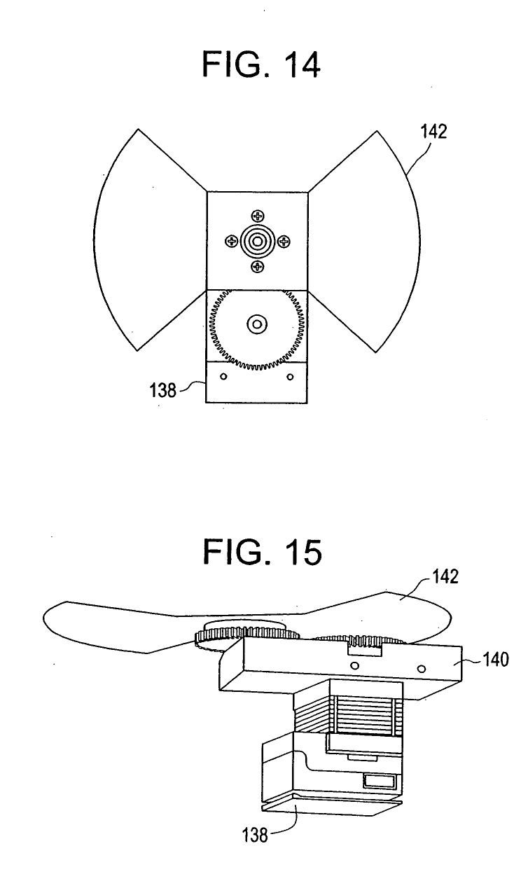

FIG. 14 illustrates a front view and FIG. 15 a top view of a chopper wheel device according to some embodiments of the present invention. The chopper-wheel device effects a “shutter” effect to the radiation. As shown, the chopper-wheel device may include a motor 138, a mounting bracket/plate 140, and a “bow-tie” disk 142. FIG. 10 illustrates that upon rotation of the bow-tie disk, the shutter effect of UV radiation is produced from the UV lamp—i.e., portions of the bow-tie disk which lack material allow radiation to pass through opening 114, while the remaining portions block radiation. Accordingly, the chopper-wheel mechanism provides a time-metered exposure of the blood in the exposure chamber. Specifically, the chopper wheel provides alternative “open” and “closed” positions of the aperture between the UV lamp and the exposure chamber. The chopper wheel/aperture device timing may be determined by using a particular synchronous motor and gear drive selected for this application. According, due to the specifications of such components, timing is highly accurate and typically only change as a result of a major malfunction. In an embodiment, the chopper device is a belt drive chopper device.

In some embodiments of the present invention, the chopper-wheel assembly preferably includes a parking device which parks the chopper-wheel in a position which substantially blocks radiation—i.e., the solid portion of the chopper-wheel block opening 114. This feature performs as an added safety feature upon shutdown of the system, so that radiation is blocked from being transmitted to the exposure chamber. Thus, during such a system shutdown, the rotating chopper-wheel automatically stops in a position such that none of the opens areas of the chopper-wheel overlap with opening 114 and/or opening 73 of the lamp housing.

The UBI system according to some embodiments of the present invention is preferably designed to provide fail-safe electrical and mechanical operation so as to ensure that blood components are not damaged and that the patient is not placed in jeopardy. This may be accomplished by controlling and monitoring various system parameters (as indicated above), which may be necessary in order to ensure a safe and therapeutically effective medical procedure. The control logic may (e.g., electronics-hardware and/or software) categorize the instrument into five (5) functional states: three (3) of which may be operational, an alert state and a fail-safe state. Transition from one state to another may be based upon sensory information obtained from various sensors monitoring the various components of the system.

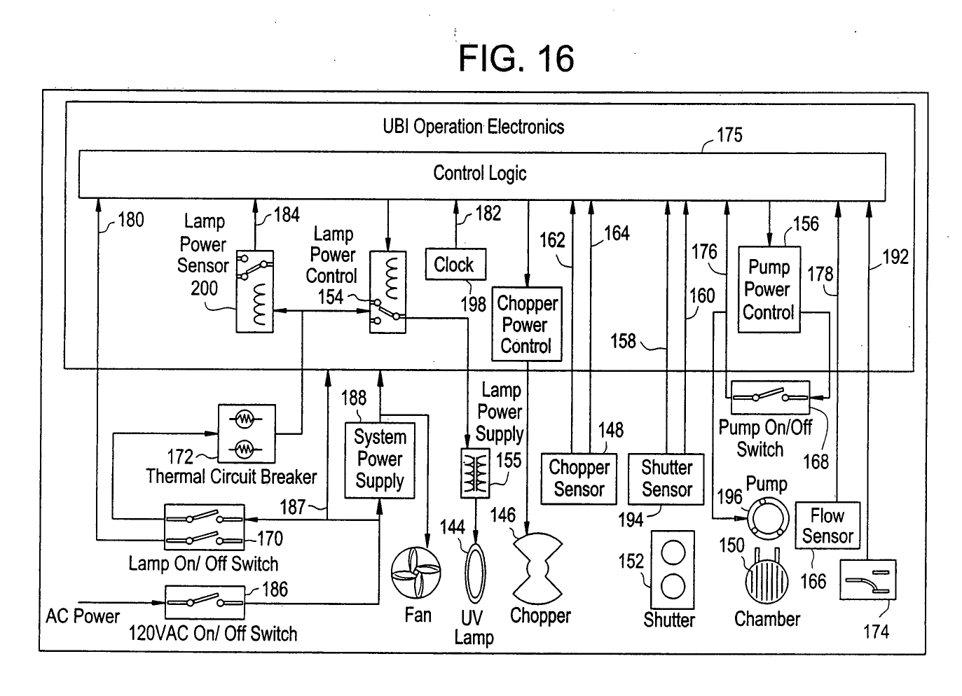

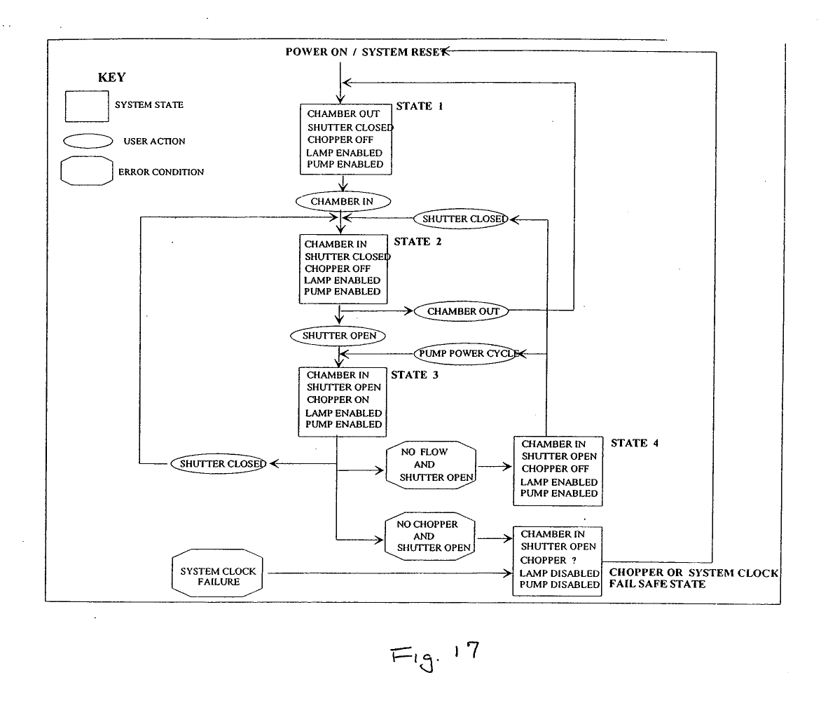

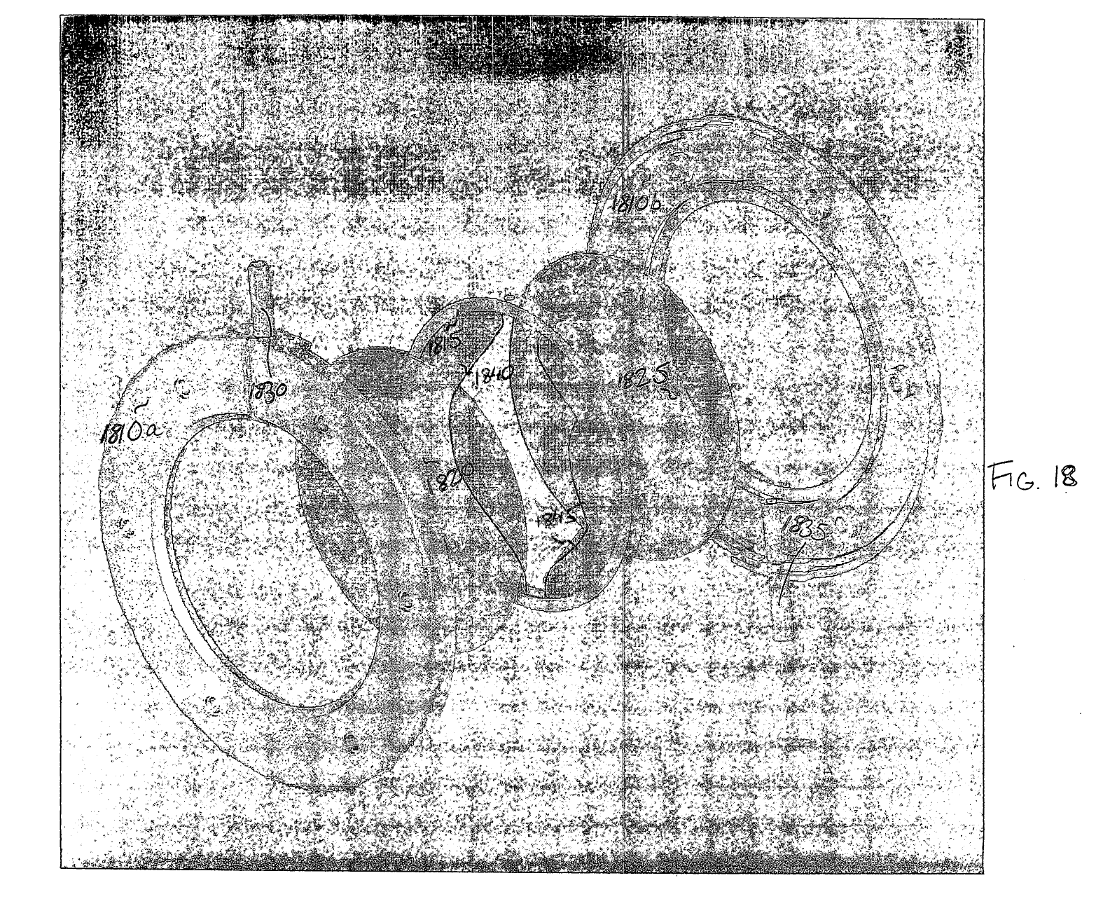

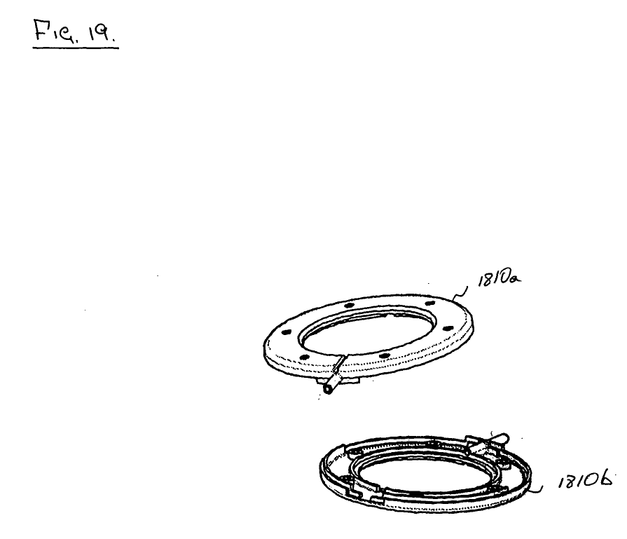

FIG. 16 depicts a block diagram of the above-described system and components thereof, as well as additional components for the control and/or monitoring of such components and the UBI system in general. Accordingly, UV lamp 144 emits a particular wavelength(s), which, after passing through chopper wheel assembly 146 and the aperture of shutter 148, enters exposure chamber 150. To accomplish this, preferably several conditions may be satisfied according to the system functions described by associating the components of FIG. 16 with the Operational States of FIG. 17.

State 1: A state in which either one or more (preferably all) of the following states occur:
chamber 150 is not inserted into the system,
shutter 152 is closed,
chopper wheel 146 is off,
UV lamp power control 154 is energized, and
pump power control 156 is energized.

State 2: A state in which either one or more (preferably all) of the following states occur:
chamber 150 is inserted into the system,
shutter 152 is closed,
chopper-wheel 146 is off,
UV lamp power control 154 is energized, and
pump power control 156 is energized.

State 3: A state in which either one or more (preferably all) of the following states occur:
chamber 150 is inserted into the system;
shutter 152 is open;
chopper-wheel 146 is on;
UV lamp power control 154 is energized; and
pump power control 156 is energized.

State 4: A state in which either one or more (preferably all) of the following states occur:
chamber 150 is inserted into the system,
shutter 152 is open, the chopper 146 is off,
UV lamp power control 154 is energized,
pump power control 156 is energized, and
either the pump On/Off switch 168 is off or the flow sensor 166 indicates No Flow.

Fail-Safe State: A state in which either one or more (preferably all) of the following states occur:
chamber 150 is inserted into the invention,
shutter 152 is open,
chopper 146 is in an unknown condition,
UV lamp power control 154 is de-energized, and
pump power control 156 is de-energized.

The following signal-sensory information (see FIG. xx) may be preferably used by the control logic to determine the appropriate state of operation.

| Signal Monitoring | Fig. xx Item |
|---|---|
| 1- Shutter fully open sensor | 158 |
| 2- Shutter fully closed sensor | 160 |
| 3- Chopper first position sensor | 162 |
| 4- Chopper second position sensor | 164 |
| 5- Flow sensor | 166 |
| 6- Pump Switch | 168 |
| 7- Lamp Switch | 170 |
| 8- 120 V AC lamp thermal breaker | 172 |
| 9- Chamber position sensor | 174 |

Based upon the status of one or more of such signals, relevant system status information may be provided to the operator and/or monitoring system—e.g., computer. These status indicators, using audio and visual means, may fall into two modes: (1) an alert mode where an alert is provided to inform a operator of an operator procedural error, and (2) an alarm mode, which provides a highly visual and/or highly audible alarm of serious instrument hardware (and/or software) malfunction, which may cause the control logic 175 to force the instrument into a fail-safe condition.

| | Signal Item | Action |
|---|---|---|
| Alert Mode | | |
| 1- No Flow and Shutter open | 176, 178, 160 | turn off |
| | | chopper-wheel |
| 2- Lamp switch off and shutter open | 180, 160 | |
| 3- Shutter not fully open or fully closed | 158, 160 | |
| Alarm Mode | | |
| 4- No Chopper motion and Shutter open | 162, 164, 160 | turn off lamp |
| | | and pump |
| 5- System clock failure | 182 | turn off lamp |
| | | and pump |
| 6- Thermal switch failure | 184 | turn off lamp |

In preferred embodiments of the invention, control logic 175 may determine the operational state of the invention at most (preferably) all times. For example, following power on of the instrument, in which AC switch 186 is activated, 120 VAC 187 (for example) is routed within the instrument to power supply 188, operation electronics and lamp On/Off switch 170. Thus, Control logic 175 preferably forces the operational condition to be in State 1.

According to some embodiments of the present invention, as part of a normal medical procedure to expose a patient's blood to UV radiation, chamber 150 may first be inserted into the system. As seen in FIG. 17, the transition from State 1 to State 2 is initiated by the insertion of the exposure chamber 150 into the system. Chamber sensor 174 confirms its proper inserted position by sending signal 192 to the control logic 175, which in turn performs the change to State 2. Removing the chamber 150 from the system (i.e., chamber receiving housing) results in an immediate return to State 1.

To continue the process of blood irradiation, the aperture (shutter mechanism) 152 is opened by manual action of the instrument operator. This is preferably done to allow the chamber 150 contents to be exposed to the UV lamp 151 radiation. The transition from State 2 to State 3 may be initiated by this action of opening the aperture 152. Shutter sensor(s) 194 may determine whether the shutter 152 is fully closed or fully open, via signals 158 and 160. If the sensor 160 which senses a fully closed status of the aperture indicates that the aperture is not fully closed (i.e. the aperture is partially open), the control logic 175 forces the instrument to be in State 3. If sensor 158, which senses the aperture being fully opened, does not indicate a fully open state, then a simple alert (#3) may be issued to inform the operator of the system that the aperture is partially open. Closing the aperture 152 fully may preferably cause the control logic 175 to force the instrument to return to State 2. It is important to note that if the chamber 150 is not inserted properly into the system at the start of the process, a safety feature of the aperture mechanism 152 preferably prevents the aperture from being opened (even partially), and hence transition from State 2 to State 3 is also thereby prevented.

As the medical procedure continues in State 3, the blood is preferably pushed through the chamber 150 by pump 196. To determine if this is occurring, the sensor 168 monitors IV tubing for an indication of flow, and the status of pump switch 168 output is determined to establish whether the switch is in an ‘on’ or ‘off’ position. If either of these conditions determines that the blood is not moving through the chamber, a “no flow” condition is preferably declared. It is worth noting that such a “no flow” condition preferably results in the control logic 175 forcing a transition to State 4. Power cycling the pump (e.g., the pump switch 168 from on to off to on), preferably causes the control logic 175 to make a system transition back to State 3. Closing aperture 152 preferably causes the control logic 175 to make a system transition back to State 2.

While in State 3, the optical aperture interrupter chopper wheel 146 may be activated. When activated, the chopper wheel preferably rotates at a specific RPM. The rotation causes a periodic “on” and “of” timing characteristic to the chamber 150 irradiation. The wheel motion may be continuously measured by sensor 148, which preferably monitors two specific locations along the circumference of the wheel. In particular, sensor 148 may forward position signals 162 and 164 to the control logic 175. The timing of this wheel rotation is preferably measured to ensure a proper exposure time for the blood flowing through the chamber 150. If the chopper wheel 146 motion stops, the control logic 175 may receive signals 162 and/or 164 from sensor 148 indicating such failure. As a result, the control logic 175 may activate an alarm (#4) to notify the operator of hardware malfunction, and also preferably deactivate both the lamp power control 154 and the pump power control 156. If sensor 148 malfunctions, chopper wheel 146 motion cannot be determined. In this failure situation also, the control logic 175 may activate an alarm (#4) and deactivate both the lamp power control 154 and the pump power control 156. These two failure conditions preferably cause the control logic 175 to force the instrument into the fail-safe state. In preferred embodiments, one way to escape from this state is to remove power to the instrument by deactivating AC Switch 186, and repair the failed item.

Also in some of the preferred embodiments, as an additional safety feature, the control logic 2 ensures that the chopper wheel 146 rests in a specific physical orientation—blocking the optical aperture between the lamp 144 and the chamber 150, when it is parked in its stopped position. Such a parking orientation may be forced whenever the instrument is in State

(for example). This feature provides a secondary back-up to the aperture 152, to protect the operator and/or patient from accidental UV exposure, if that mechanism is improperly forced open without the use of the specified exposure chamber 37 (for example).

In some embodiments of the invention, in all States of operation, the control logic 175 monitors the system for the occurrence of two particular types of failures. First, the control logic monitors the system for a failure of the internal system timing clock 198. Such a failure may cause the control logic 175 to initiate an immediate transition of the instrument into the fail-safe state. In particular, the Control Logic 175 may activate an alarm (#5) and deactivate both the lamp power control 154 and the pump power control 156. The second type of failure may be an overheat event which causes thermal circuit breaker 172 to “open”, thereby removing AC power from the lamp power sensor 200 and from the lamp power supply 155. In such a failure situation, the instrument may not be able to illuminate the lamp 144, and may then be un-powered, and repaired. An alarm (#6) condition may then notify the operator of this status.

Accordingly, the above embodiments enable blood (and/or other fluids) to be safely and effective irradiated. Such embodiments may be used to irradiate blood according to the following exemplary protocol. For example, subjects undergo one or more sessions of (preferably) five ultraviolet blood irradiation treatments over a three-week period.
Treatment #1 Start;
Treatment #2, within 48 hours of the prior treatment;
Treatment #3, within 72 hours of the prior treatment;
Treatment #4, within five (5) days of the prior treatment; and

Treatment #5, within five (5) days of the prior treatment.

Sample Treatment Schedule

| | Sunday | Monday | Tuesday | Wednesday | Thursday | Friday | Saturday |
|---|---|---|---|---|---|---|---|
| Week 1 | | | | Treatment 1 | | Treatment 2 | |
| Week 2 | | Treatment 3 | | | | | Treatment 4 |
| Week 3 | | | | | Treatment 5 | | |

The treatment may be accomplished by introducing a standard 20 gauge intravenous catheter into the patient's vein, and 1.5 cc of blood per pound of body weight is withdrawn according to the following formula: A=KW, where K is a constant (1.5 cc), and W is the patient's body weight in pounds. Preferably, the total amount of blood withdrawn should not exceed 250 ml in total.

The blood may be collected into a vacuum container prepared with 3000 to 5000 units of heparin sodium. The container is carefully inverted to mix the blood with the heparin, and then may be hung from an IV pole attached to the UBI system. The blood is then circulated through the exposure chamber, thereby exposing the blood to UV radiation. (e.g., UVC at between about 200 nm and about 400 nm), at a rate of approximately 30 ml/minute, before being returned to the patient.

The irradiated blood may then be returned to the patient at the fasted infusion rate allowed (per a standard administration set). A typical duration of the procedure is approximately 20 minutes.

Other embodiments of the invention may include systems for diagnostic applications with or without the use of a drug. For example, a predetermined therapy using one or another of the above disclosed system/device embodiments simulates the immune system, which initially seeks out blood borne pathogens and inflammation. A blood test at a predetermined time later may reveal and contribute to a diagnostic process. In addition, such a therapy may exacerbate an inflammatory reaction of a low grade and or an undetectable infection, which can be sighted using imaging devices, blood tests and patient feedback.

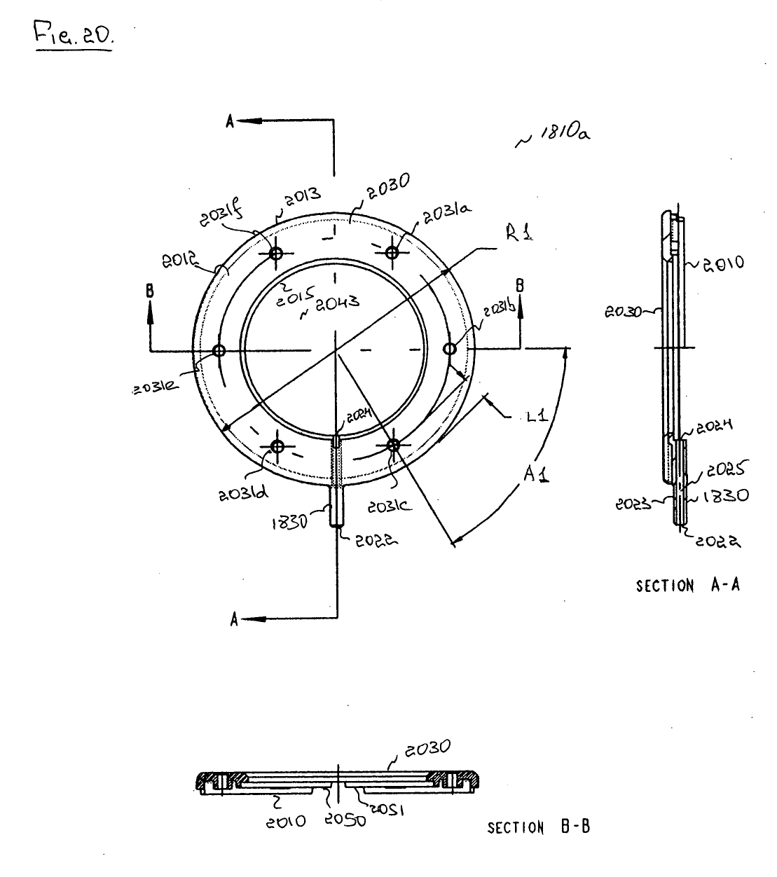

FIGS. 18-26 illustrate an exemplary embodiment of the exposure chamber 1800. FIG. 18 is an exploded perspective view of the exposure chamber 1800. The chamber 1800 includes a first housing 1810a, a second housing 1810b, a gasket 1815, a first UV filter lens 1820, a second UV filter lens 1825, a first insert 1840, and a second insert 1845. The housings 1810a, and 1810b are configured to be coupled together to form the exposure chamber 1800. The gasket 1815, filters 1820 and 1825, and inserts 1840, 1845 are configured to fit inside and between the first housing 1810a and the second housing 1810b. The first insert 1840 and the second insert 1845 are configured to be secured inside the gasket 1815. AS can be understood by one skilled in the art, there can be single or multiple inserts configured to be placed inside the gasket 1815.

The first housing 1810a includes a first conduit 1830. The second housing 1810b includes a second conduit 1835. The conduits 1830 and 1835 serve as ports for entry and exit of blood or its components to and from the exposure chamber 1800. In an embodiment, the first conduit 1830 serves as an entry port and the second conduit 1835 serves as an exit port. In an alternate embodiment, the functions of conduits 1830 and 1835 are reversed.

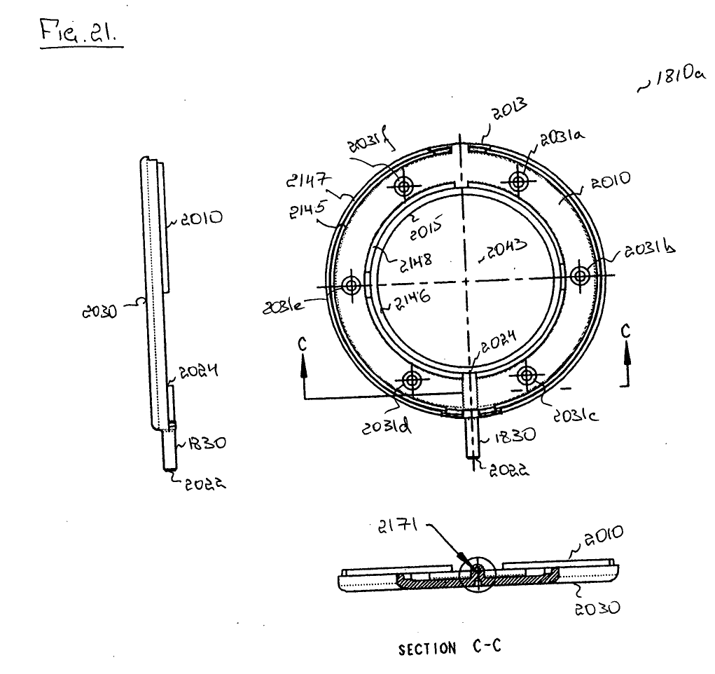

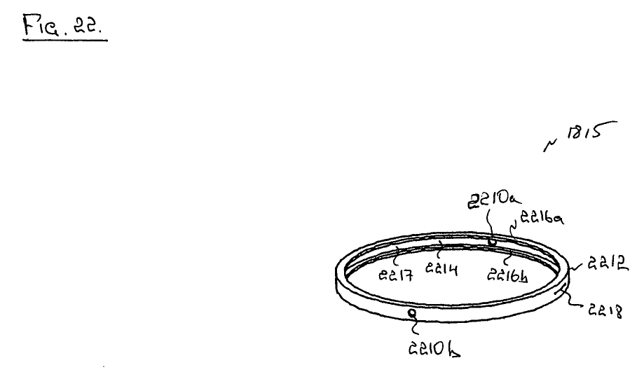

FIGS. 19-22 illustrate housings 1810a and 1810b in more detail. FIG. 20 illustrates a first housing 1810a. Since, the second housing 1810b is similar to the first housing 1810a, the description of the first housing 1810a is applicable to the second housing 1810b. FIG. 20 illustrates a top view of the first housing 1810a's outside surface 2030 and side views of the housing 1810a. The side views of the first housing 1810a are taken at directional lines AA and BB shown in the top view of the first housing 1810a. The housing 1810a has a circular or doughnut shape with an empty interior 2043. The solid portion 2012 of the housing 1810a that surrounds the empty interior 2043 is enclosed by an interior edge 2015 and an exterior edge 2013. The exterior edge 2013 has a diameter R1, as illustrated in FIG. 20. The diameter of the interior edge 2015 is less than the diameter of the exterior edge 2013.

The solid portion 2012 of the housing 1810a includes openings 2031 (a, b, c, d, e, f). The openings 2031 secure the first housing 1810a and the second housing 1810b together during assembly of the exposure chamber 1800. The housings are secured together using bolts, screws, fasteners or any other suitable means. As shown in FIG. 20, the openings 2031 are evenly spaced out throughout the outer surface 2012. This allows for a secure and tight fitting of the housings 1810a and 1810b. Specifically, each opening 2031 is separated by an angle A1 from the other opening 2031. The openings 2031 are also disposed a distance L1 from the exterior edge 2013 of the housing 1810a.

The housing 1810a includes the first conduit 1830. The conduit 1830 protrudes away from the exterior edge 2013. The first conduit 1830 includes a tube 2023 having a hollow interior 2025, an outside tip 2022 and an inside tip 2024. The outside tip 2022 is disposed outside the first housing 1810a and the inside tip 2024 is disposed on the inside surface 2010 (shown in side views in FIG. 20) at the interior edge 2015, as shown in the Section A-A view of the housing 1810a in FIG. 20. The first conduit 1830 is secured to the inside surface 2010 of the housing 1810a.

The housing 1810a also includes a grooved portion 2050, as shown in the B-B section view of FIG. 20. The grooved portion 2050 accommodates the second conduit 1835 (not shown in FIG. 20) of the second housing 1810b (also not shown in FIG. 20). The second housing 1810b includes a similar grooved portion (not shown in FIG. 20) that accommodates the first conduit 1830. In an embodiment, the grooved portion 2050 includes multiple grooves 2051, as shown in Section B-B of FIG. 20, that accommodate the conduit 1830. In an alternate embodiment, the grooves 2051 on the housings 1810a and 1810b are configured to fit the conduits 1830 and 1835, respectively, to create a sealed connection between the two housings 1810a and 1810b.

FIG. 21 illustrates an inside surface 2010 of the housing 1810a. FIG. 21 also illustrates side views of the housing 1810a, where one side view is a directional cross-section view of the housing 1810a taken at a C-C line (titled “Section C-C”) and the other side view is a plain side view of the housing 1810a. As shown in FIG. 21, the inside surface 2010 includes openings 2031 that correspond to the openings 2031 shown in FIG. 20 above. The openings 2013 go through the housing 1810a from the outside surface 2030 to the inside surface 2010. The openings 2031 have a similar structure in the second housing 1810b. The openings of the first and second housings 1810a and 1810b interact with each other to secure the two housings together during assembly of chamber 1800.

The top view of the inside surface 2010 in FIG. 21 show that the conduit 1830 and the grooved portion 2050 are disposed along the same line on the inside surface 2010 of the housing 1810a. Alternatively, the conduit 1830 and the grooved portion 2050 can be disposed at different locations on the inside surface 2010.

The inside surface 2010 of the housing 1810a includes a ledge 2145 and a side wall 2147 disposed along the exterior edge 2013. The sidewall 2147 is substantially perpendicular to and protrudes away from the inside surface 2010. The housing 1810b includes a similar ledge 2145 and a sidewall 2147 on its inside surface 2010. During assembly of the chamber 1800, the ledges and sidewalls in both housings come in contact with each other to create a secure connection between housings 1810a and 1810b. The ledges 2145 and sidewalls 2147 provide an additional security when housing 1810a and 1810b are bolted (or otherwise secured to each other) together using openings 2031. In an embodiment, to provide additional security an additional locking mechanism can be implemented to lock the housings 1810a and 1810b. Such locking mechanism can be a snap-on lock, a friction fit lock, or any other locking mechanism suitable for this purpose. The mechanism can be disposed anywhere on the housings 1810a and 1810b.

The inside surface 2010 of the housing 1810a also includes a ledge 2146 and a sidewall 2148 disposed along the interior edge 2015. The sidewall 2148 is perpendicular to and protrudes away from the inside surface 2010 of the housing 1810a. The ledge 2146 accommodates placement of UV filter lens 1820 and gasket 1815 (similarly for the housing 1810b, where its ledge 2146 accommodates placement of UV filter lens 1825 and gasket 1815).In an embodiment, the sidewall 2148 has a thickness that is substantially equal to the combined thickness of UV filter lens 1820 and at most half of the thickness of the gasket 1815. The design of ledge 2146 and sidewall 2148 in both housings 1810a and 1810b allows for a secure and tight assembly of the chamber 1800.

The Section C-C cross-sectional view of FIG. 21 illustrates an opening 2171 in the conduit 1830. The size of the opening 2171 is specific to the design of chamber 1800 as well as based on the specification of the system described in FIGS. 1-17 above.

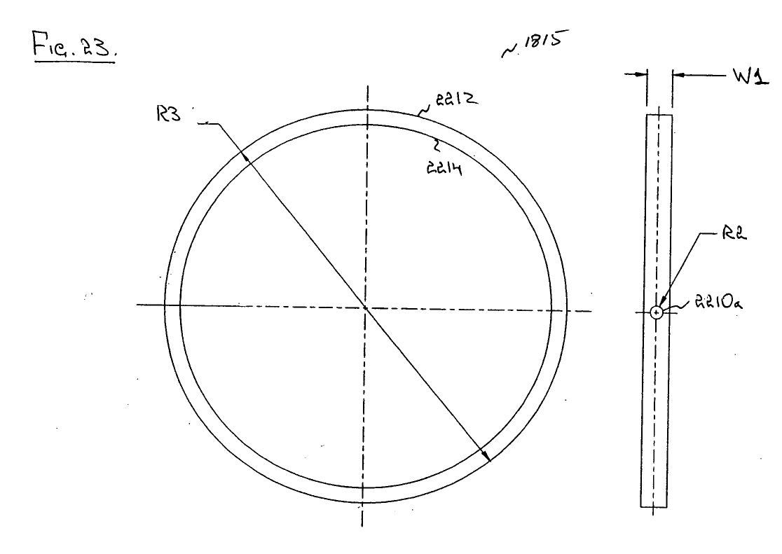

FIG. 22 illustrates the gasket 1815 of the exposure chamber 1800. The gasket 1815 includes an outer rim 2212 and an inner rim 2214. The inner rim 2214 includes sidewalls 2216a and 2216b. The sidewalls 2216 are disposed along the circumference of the inner rim 2214. The sidewalls 2216 form a groove 2217. The groove 2217 is disposed along the circumference of the inner rim 2214 of the gasket 1815. The groove 2217 is configured to fit inserts 1840 and 1845, which are described with respect to FIG. 25 below. The thickness of the groove 2217 allows for secure placement of the inserts of 1840 and 1845. The inserts 1840 and 1845 can be also glued, welded, friction fit, or otherwise further secured inside groove 2217 and between sidewalls 2216a and 2216b.

The gasket 1815 further includes a slit 2218. The slit 2218 is disposed approximately midway in the outer rim 2212 and extends through the gasket's walls into the groove 2217. The slit 2218 is configured to accommodate a protrusion 2512 (not shown in FIG. 22) of the insert 1840, as described below with respect to FIG. 25.

The gasket 1815 further includes openings 2210a and 2210b. The openings 2210 are disposed in the outer rim 2212 of the gasket 1815 and extend through the walls of the gasket into the groove 2217. As such, the openings 2210 provide a connection between the outer and inner portions of the gasket 1815. The openings 2210 are further configured to coincide with the openings in the first and second conduits 1830 and 1835 (not shown in FIG. 22). FIG. 22 further illustrates that the openings 2210 are disposed diametrically opposite of each other. As can be understood by one skilled in the art, the openings 2210 can be disposed anywhere on the gasket's outer rim 2212, as long as the openings 2210 coincide with conduits 1830 and 1835.

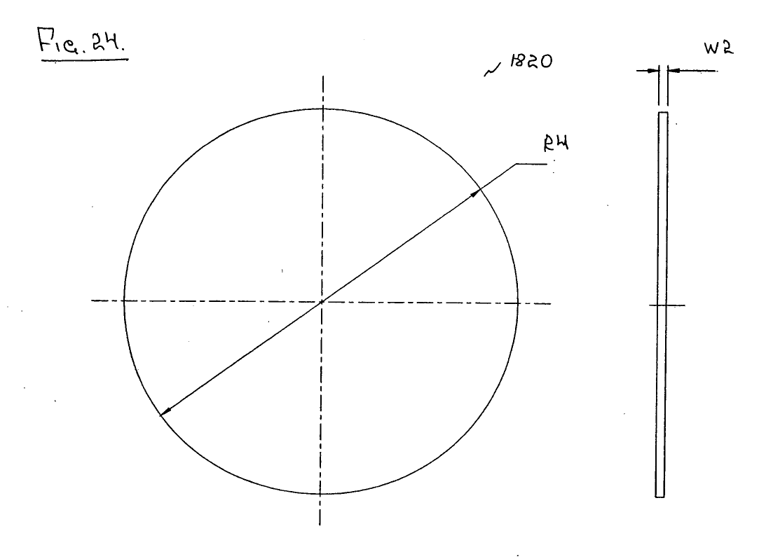

FIG. 23 illustrates top and side views of the gasket 1815. The side view of the gasket 1815 shows the opening 2210a in the outer rim 2212 of the gasket 1815. In an embodiment, a diameter R2 of the opening 2210a is equal to 0.078 inches. A diameter R3 of the gasket 1815 is equal to 2.3 inches. The width W1 of the outer rim 2212 is equal to 0.15 inches. As can be understood by one having ordinary skill in the art, other configurations of gasket 1815 are possible.

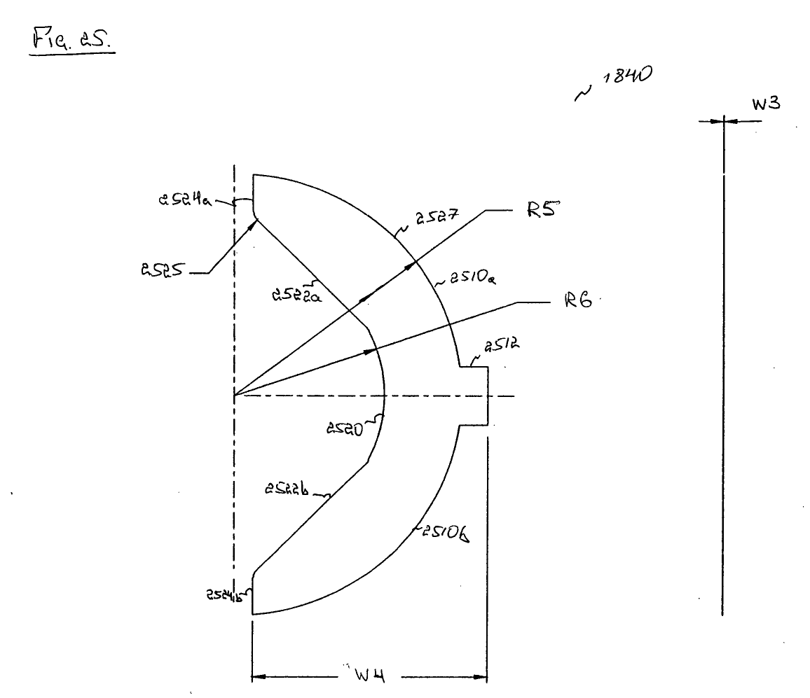

FIG. 24 illustrates a first UV filter lens 1820. The filter lens 1820 has a diameter R4 and a thickness W2. In an embodiment, R4 is equal to 2.25 inches and W2 is equal to 0.05 inches. The first filter lens 1820 is configured to be placed between the gasket 1815 and the first housing 1810a and the second filter lens 1825 is configured to be placed between the gasket 1815 and the second housing 1810b. As stated above, the ledges 2146 and sidewalls 2148 of the housings 1810a and 1810b secure the filter lenses 180 and 1825 to the respective housings 1810a and 1810b.

FIG. 25 illustrates a first insert 1840. The insert 1840 has a thickness of W3. In an embodiment, the thickness W3 is equal to 0.001 inches.

The insert 1840 has a multi-contoured inner surface 2525 and a substantially round outer surface 2527. The outer surface 2527 includes two round portions 2510a and 2510b separated by a protrusion 2512. The round portions 2510 have a radius R5. In an embodiment, the radius R5 is equal to 1.125 inches. The round portions 2510 and the protrusion 2512 are configured to fit inside the inner rim 2214 of the gasket 1815. Specifically, the insert 1840 is configured to fit between the sidewalls 2216a and 2216b of the gasket 1815. Also, the protrusion 2512 is configured to fit inside the slit 2218 of the gasket 1815. This allows the gasket 1815 to secure the insert 1840 to its inner rim 2214. Further, the round portions 2510 of the insert 1840 are configured to also fit inside the gasket 1815. In an embodiment, the radius R5 of the round portions 2510 is substantially equal to the radius of the inner rim 2214 of the gasket 1815.

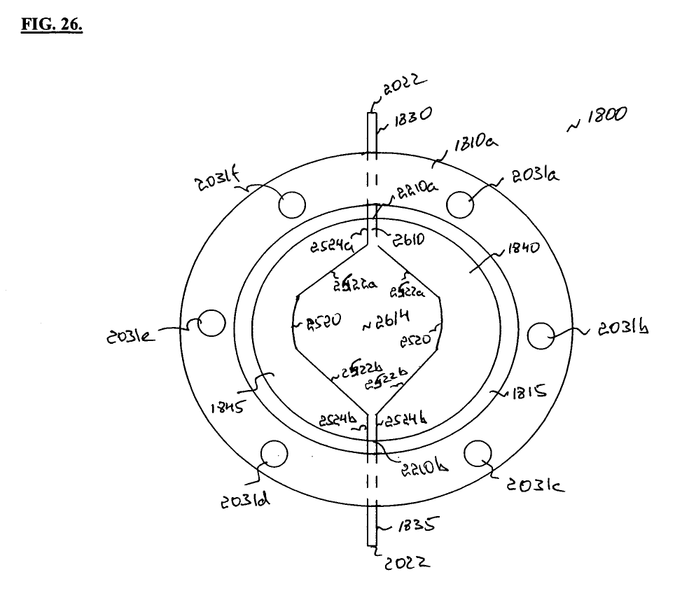

The inner surface 2525 includes a middle portion 2520 that is adjacent to two side portion portions 2522a and 2522b. The side portions 2522a, 2522b are adjacent to two edge portions 2524a and 2524b, respectively. The middle portion 2520 is round and has a radius R6. In an embodiment, R6 is equal to 0.740 inches. The two side portions 2522 are substantially straight and extend away from the middle portion 2520 and towards the edge portions 2524. The edge portions 2524 of the insert 1840 are substantially straight and are configured to be parallel to edge portions of the insert 1845 when inserts 1840 and 1845 are placed inside the gasket 1815 opposite the each other, as shown in FIG. 26 below.

The insert 1840 has a width W4 measuring from the end of the protrusion 2512 to the edges 2524. In an embodiment, the width W4 is equal to 1.156 inches. When both inserts 1840 and 1845 are secured inside the gasket 1815, the inserts form a gap, as shown in FIG. 26. The gap is formed between the edges 2524, side portions 2522, and the middle portions 2520 of each insert. The gap formed by the edges 2524 of the inserts 1840 and 1845 also coincides with the openings 2210 of the gasket 1815.

When completely assembled, the chamber 1800 forms a micro-channel 2610, as shown in FIG. 26. The micro-channel 2610 includes hollow interiors of the first and second conduits 1830 and 1835, gasket openings 2210a and 2210b, and the gap formed by the first and second inserts 1840 and 1845 inside the gasket 1815.

The blood or any other liquid that flows through the micro-channel 2610 enters at an entry point coinciding with the outside tip 2022 of the conduit 1830. Then, it proceeds through the hollow portion of the conduit 1830 to its inside tip 2024. After that it continues to flow through the opening 2210a in the gasket 1815. Then, it enters the gap formed by the inserts 1840 and 1845 inside the gasket 1815. Once the liquid entered into the gap, it spreads to the inner surfaces 2525 of the inserts 1840 and 1845. After that, it continues to flow through the opening 2210b in the gasket 1815. It then enters the hollow portion of the conduit 1835 at its inside tip 2024. It continues to flow through the hollow portion of the conduit 1835 to its exit point 2022. The tips of the conduits 1830 and 1835 are coupled to blood pumping and reservoir devices as well as other components of the blood irradiation system described above with respect to FIGS. 1-17.

The blood is exposed to UV light radiation, when it is pumped through the micro-channel 2610. The actual exposure takes places in an open area or exposure area 2614. The open area 2614 is formed by the inserts 1840 and 1845, as shown in FIG. 26. FIG. 26 illustrates an assemble exposure chamber 1800.

When blood enters the open area 2614, the UV lamp is activated, as described above with respect to FIGS. 1-17, and the blood is exposed to UV light radiation. After exposure, the blood exits the micro-channel 2610 through the conduit 1835.

As stated above, the exposure chamber 1800 is configured to be coupled to a pumping device and blood reservoir capable of pumping the blood in and out of the micro-channel channel 2610. In an embodiment, the width of the micro-channel 2610 is equal to 0.010 inches. In an alternate embodiment, the width of the micro-channel 2610 is less than 0.005 inches. In another alternate embodiment, the width of the micro-channel 2610 is in a range between 0.0005 inches and 0.002 inches.

The micro-channel 2610 allows a thin-film like flow of blood through the exposure chamber 1800. As can be understood by one skilled in the art, the whole blood or its components such as platelets, red cell constraints, factor VIII & IX, or other components can be used for exposure. In an alternate embodiment, the blood can be diluted with PBS and anti-coagulates. Blood can be a human blood, an animal blood, or any other liquid.

The gasket member 1815 is manufactured from a biocompatible metal or plastic or any other suitable material.

In an embodiment, the flow rate of blood through the exposure chamber 1800 is between about 0.1 to 5 ml per minute. In an alternate embodiment, the flow rate can be about 1 ml per minute. In another alternate embodiment, the flow rate is in a range of 10 to 15 ml per minute. The flow can be produced by a vacuum (for example, 10-20 mm Hg) to minimize hemolysis. The flow rate can be increased by increasing the lens size and irradiation level.

While certain embodiments of the present invention have been herein described, such descriptions have been provided as examples only and not as limitations to the invention. Accordingly, as one of ordinary skill in the art will appreciate, numerous other embodiments, some with additional or less features, are within the scope of this invention, a few embodiments of which are hereinafter claimed.

What is claimed:

1. An exposure chamber for exposing blood to radiation in a blood irradiation system having an ultraviolet UV source, a connector between the UV source and the exposure chamber, a pump for pumping blood through the exposure chamber, and a shutter assembly provided between the UV source and the exposure chamber for time-metered radiation of the blood in the exposure chamber, where the exposure chamber comprises
a housing including an entry conduit and an exit conduit;
a UV filter lens configured to be secured within said housing;
a gasket configured to be secured within said housing and substantially adjacent to said UV filter lens, said gasket includes openings configured to be in communication with said entry conduit and said exit conduit of said housing;
wherein said gasket includes an insert configured to create an exposure area, said exposure area is further configured to be in communication with said openings of said gasket;
said entry and exit conduits, said openings, and said exposure area are configured to create a channel for permitting blood flow through the exposure chamber.

2. The chamber of claim 1, where said gasket includes another insert and another opening;
said another opening is configured to be in communication with said exit conduit.

3. The chamber of claim 2, wherein said insert and said another insert include multi-contoured edges and are configured to be secured within said gasket, wherein said multi-contoured edges are further configured to create said exposure area within said gasket;
said exposure area is configured to allow blood exposure to UV light radiation when blood flows through said channel.

4. The chamber of claim 3, wherein said channel is configured to allow blood flow through
said entry conduit;
said opening in said gasket;
said exposure area;
said another opening in said gasket; and
said exit conduit.

5. The chamber of claim 1, wherein said channel is configured to allow blood flow at a rate in a range of 0.1 to 5 milliliters per minute.

6. The chamber of claim 1, wherein said channel is configured to allow blood flow at a rate in a range of 10 to 15 milliliters per minute.

7. The chamber of claim 1, wherein said channel is configured to allow blood flow at a rate of 1 milliliter per minute.

8. The chamber of claim 1, wherein blood flowing through the exposure chamber is a component of blood.

9. The chamber of claim 1, wherein a width of said channel is 0.01 inches.

10. The chamber of claim 1, wherein a width of said channel is less than 0.005 inches.

11. The chamber of claim 1, wherein a width of said channel is in a range of 0.0005 to 0.002 inches.

12. The chamber of claim 1, wherein said UV filter lens is configured to prevent leakage of blood from the exposure chamber when blood flows through said channel.

13. An exposure chamber for exposing blood to radiation in a blood irradiation system having an ultraviolet UV source, a connector between the UV source and the exposure chamber, a pump for pumping blood through the exposure chamber, and a shutter assembly provided between the UV source and the exposure chamber for time-metered radiation of the blood in the exposure chamber, where the exposure chamber comprises
a housing including a conduit;
another housing including another conduit, wherein said housing is configured to be coupled to said another housing;
a gasket configured to be secured between said housing and said another housing and including openings configured to be aligned with said conduit and said another conduit;
a filter lens configured to be secured between said housing and said gasket;
another filter lens configured to be secured between said another housing and said gasket;
an insert and another insert configured to be secured within said gasket, said inserts form an exposure area within said gasket;
said conduits, said openings and said exposure area form a channel configured to allow blood to flow through said exposure chamber.

14. The chamber of claim 13, wherein blood flows through said channel at a rate in a range of 10 to 15 milliliters per minute.

15. The chamber of claim 13, wherein blood flows through said channel at a rate in a range of 0.1 to 5 milliliters per minute.

16. The chamber of claim 13, wherein blood flows through said channel at a rate of 1 milliliter per minute.

17. The chamber of claim 13, wherein blood is exposed to UV light in said exposure area.

18. The chamber of claim 17, wherein said channel is configured to have an unexposed blood enter the exposure chamber through said conduit and to have an exposed blood exit the exposure chamber through said another conduit.

19. The chamber of claim 13, the blood flows through said channel under pressure [symbol] ted by a pumping device configured to be coupled to the exposure chamber.

20. The chamber of claim 13, wherein said housings and said filter lenses are configured to prevent escape of blood from said channel.

21. The chamber of claim 13, wherein said insert and said another insert include multi-contoured edges and are configured to be secured within said gasket, wherein said multi-contoured contoured edges are further configured to create said exposure area within said gasket;
said exposure area is configured to allow blood exposure to UV light radiation when blood flows through said channel.

22. The chamber of claim 13, wherein blood flowing through the exposure chamber is a component of blood.

23. The chamber of claim 13, wherein a width of said channel is 0.01 inches.

24. The chamber of claim 13, wherein a width of said channel is less than 0.005 inches.

25. The chamber of claim 13, wherein a width of said channel is in a range of 0.0005 to 0.002 inches.

26. A micro-channel assembly for a blood irradiation device having an exposure chamber, where the micro-channel comprises
a conduit configured to be secured within a housing of the exposure chamber;
an opening within a gasket of the exposure chamber and configured to be in communication with said conduit;
an exposure area within said gasket and configured to communicate with said opening;
another opening within said gasket and configured to communicate with said exposure area;
another conduit secured within said housing and further configured to communicate with said another opening.

27. The micro-channel of claim 26, wherein the micro-channel channel is configured to allow blood flow through
said entry conduit;
said opening in said gasket;
said exposure area;
said another opening in said gasket; and
said exit conduit.

28. The micro-channel of claim 26, wherein blood flows through said micro-channel at a rate in a range of 10 to 15 milliliters per minute.

29. The micro-channel of claim 26, wherein blood flows through said micro-channel at a rate in a range of 0.1 to 5 milliliters per minute.

30. The micro-channel of claim 26, wherein blood flows through said micro-channel at a rate of 1 milliliter per minute.

31. The micro-channel of claim 26, wherein blood flowing through the micro-channel is a component of blood.

32. The micro-channel of claim 26, wherein a width of said micro-channel is 0.01 inches.

33. The micro-channel of claim 26, wherein a width of said micro-channel is less than 0.005 inches.

34. The micro-channel of claim 26, wherein a width of said micro-channel is in a range of 0.0005 to 0.002 inches.
